(19) United States
(12) Patent Application Publication
(10) Pub. No.: US 2007/0016248 A1
(43) Pub. Date: Jan. 18, 2007

(54) Medical gripping and/or cutting instrument

(76) Inventors: Alfred Cuschieri, St. Andrews (GB); Timothy Graham Frank, Wormit Newport-On-Tay (GB); Stuart I. Brown, St. Andrews (GB)

Correspondence Address: ST. ONGE STEWARD JOHNSTON & REENS, LLC, 986 BEDFORD STREET, STAMFORD, CT, 06905-5619, US

(21) Appl. No.: 11/452,521

(22) Filed: Jun. 14, 2006

(30) Foreign Application Priority Data
Jun. 14, 2005 (DE) 10 2005 027 418.8

(51) Int. Cl. A61B 17/00

(52) U.S. Cl. 606/205

(57) ABSTRACT

The invention relates to a medical gripping and/or cutting instrument with a shaft having on its distal end a tool consisting of at least two jaw members and on its proximal end a handle consisting of at least two gripping members. For opening and closing, at least one jaw member of the tool can be displaced with respect to the other jaw member of the tool by means of a rotatably configured gripping member of the handle. To produce a medical gripping and/or cutting instrument that allows operation that relieves the hand along with optimized action, it is proposed with the invention that at least one portion of at least one gripping member of the handle can rotate around the longitudinal axis of this gripping member.

### FIELD OF THE INVENTION

The invention relates to a medical gripping and/or cutting instrument having a shaft on whose distal end a tool consisting of at least two jaw members is positioned and on whose proximal end a handle consisting of at least two gripping members is positioned. For opening and closing, at least one jaw member of the tool can be displaced with respect to the other jaw member by means of one rotatably configured gripping member of the handle.

### BACKGROUND OF THE INVENTION

Generic medical instruments are known in the art in various embodiments. Because a piece, such as a surgical needle, which is gripped by means of the jaw members of the tool, must often be fixed firmly between the jaw members of the tool for an extended time period, the operator is required to hold both of the gripping members of the handle pressed together continuously. During lengthier tasks the gripping members of the handle can rub so strongly against the palm of the user's hand as to cause blisters to form. In addition to the possibility of blister formation, medical instruments known in the art have the disadvantage that the gripping position does not always ensure the best possible transmission of power from the hand to the instrument.

Consequently it is the object of the invention to produce a medical gripping and/or cutting instrument that allows operation that protects the hand while also optimizing the action.

### SUMMARY OF THE INVENTION

The solution of this objective by means of the invention is distinguished in that at least one partial area of at least one gripping member of the handle can rotate around the longitudinal axis of this gripping member.

Because the invention includes the formation of a portion of a handle as a partial area that can swivel around the longitudinal axis of the gripping member, it is possible for the first time to vary the position of the handle in the hand without releasing or regripping the handle, in such a way that, first, no pressure points are exerted on the operator's hand and, second, an always optimal position of the gripping members toward the operator's hand and finger position is guaranteed in order to allow the best possible transmission of force from the hand to the instrument.

According to a practical embodiment of the invention it is proposed that a gripping member of the handle is of bipartite construction, consisting of a rigid portion and a rotatable portion, so that the rotatable portion can rotate at least partially around the rigid portion. This bipartite nature of the gripping member is advantageous as an alternative to the configuration of a gripping member as totally rotatable around its longitudinal axis, because the rigid portion of the construction lends the instrument the stability necessary for the intended handling.

To be able to grip the rotatable portion of the gripping member securely and to facilitate rotation, it is proposed according to the invention that gripping indentations are formed on the rotatable portion of the gripping member.

It is further proposed with the invention that the angle of rotation of the rotatable portion can be restricted by stops in order to prevent the gripping member from slipping in the operator's hand.

Finally, it is proposed with the invention that the gripping members of the handle are pretensioned in the open position by means of a spring element. This pretensioning of the gripping members serves to move the gripping members of the handle into the open position without any effort by the operator.

Additional characteristics and advantages of the invention can be seen from the appended illustrations, in which an embodiment of a medical gripping and/or cutting instrument according to the invention is presented in merely exemplary manner.

### BRIEF DESCRIPTION OF THE DRAWINGS

FIG. 1 shows a side view of a medical gripping and/or cutting instrument according to the invention, in a first working position of the gripping members.

FIG. 2 shows a rear view of the depiction in FIG. 1.

FIG. 3 shows a side view of the medical gripping and/or cutting instrument according to FIG. 2, but showing the gripping members in a second working position.

FIG. 4 shows a rear view of the depiction in FIG. 3.

### DETAILED DESCRIPTION OF THE INVENTION

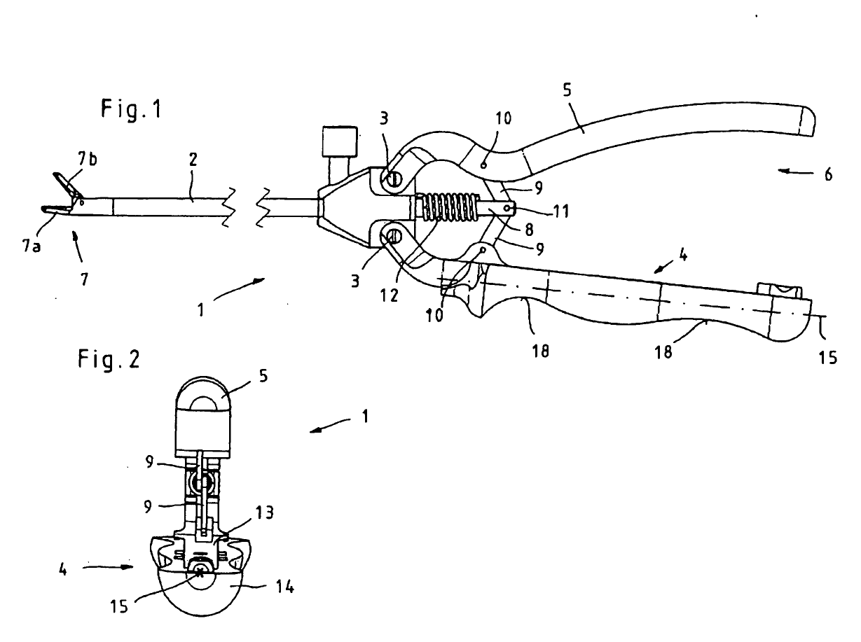

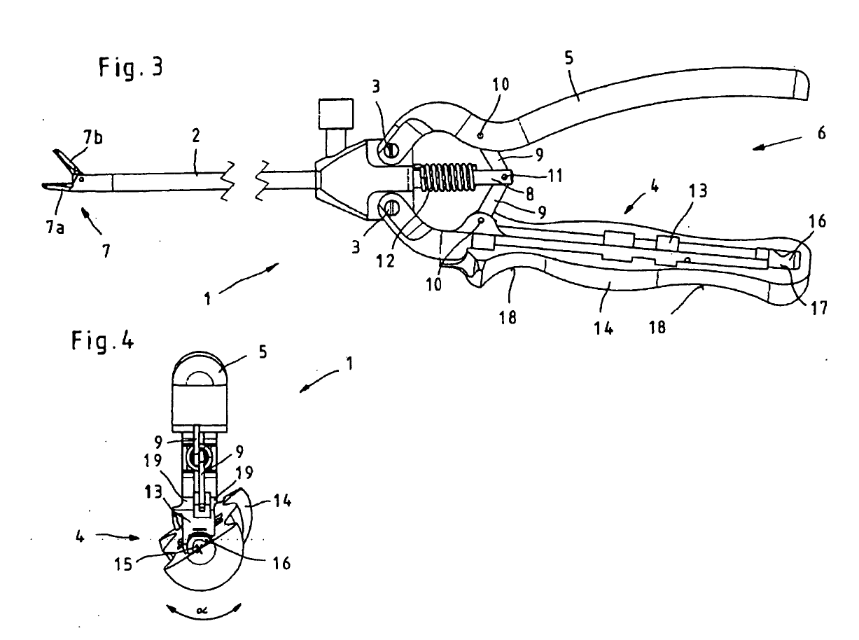

FIGS. 1 and 3 show lateral sectional views of a medical gripping and/or cutting instrument 1 whose power transmission mechanism can be put to many uses, for example as a punch, scissors, needle holder, gripping instrument, and the like.

The illustrated medical instrument consists essentially of a hollow shaft 2 on whose proximal end a handle 6 is positioned which consists of two gripping members 4 and 5 that can rotate around their respective rotation axes 3. On the distal end of the shaft 2 a tool 7 is positioned which in the illustrated embodiment consists of a jaw member 7a rigidly connected with the shaft 2, and a rotatable jaw member 7b. To open and close the jaw members 7a and 7b of the tool 7 by means of the activation of the rotatable gripping members 4 and 5 of the handle 6, the gripping members 4, 5 and the rotatable jaw member 7b are actively connected with one another by means of a push-pull rod 8.

As can be seen from FIGS. 1 and 3, the gripping members 4 and 5 are coupled with the push-pull rod 8 in the illustrated embodiment by interposed swivel levers 9, which are positioned with one free end rotatable on a support point 10 on one of the gripping members 4, 5 and with the other free end on a common axis of rotation on the push-pull rod 8. The linkage of the gripping members 4 and 5 to the push-pull rod 8 by way of the swivel lever 9 is structured in such a way that upon pressing together the gripping members 4, 5, the push-pull rod 8 is pulled by the swivel lever 9 to the proximal end of the medical instrument 1, causing the rotatable jaw member 7b of the tool 7 to rotate into the closed position

To open the jaw members 7a, 7b of the tool 7 thus requires the gripping members 4, 5 of the handle 6 to move apart, so that the push-pull rod 8 is pressed by the swivel lever 9 to the distal end of the medical instrument 1. To facilitate the pressing apart of the gripping members 4, 5 of the handle 6, a spring element 12 in the form of a draw spring is positioned on the push-pull rod 8 and serves to pretension the gripping members 4, 5 into the open position.

To allow the operator, even without releasing or regripping the gripping members 4 and 5 of the handle 6, to alter the position of the gripping members 4 and 5 in the hand, the gripping member 4 is of bipartite construction, consisting, that is, of one rigid portion 13 and a portion 14 that can rotate with respect to the rigid portion 13, as can be seen in particular from FIG. 4. In the illustrated embodiment the movable portion 14 is positioned on the rigid portion 13 of the gripping member 4 so that it can rotate around the longitudinal axis 15 of the gripping member 4, and the portions 13 and 14 are positioned mutually to one another by mounting pins 16 formed on the rigid portion 13, which pins engage with mounting ports 17 on the rotatable portion 14.

As can be seen from FIGS. 1 and 3, gripping indentations are made on the rotatable portion 14 of the gripping member 4, serving to insert the fingers of the hand holding the instrument, thus facilitating the gripping and swiveling of the rotatable portion 14.

Alternatively to the illustrated embodiment, it is also possible of course to configure a gripping member 4 or 5 in such a manner that it is alternately divided into rigid portions 13 and rotatable portions 14, that is, for instance, the area with the proximal gripping indentation 8 is configured as rigid, while the area that has the distal gripping indentation 8 is configured as rotatable.

According to a differently constructed embodiment, it is possible to configure the entire gripping member 4 or 5 of a handle 6 of a medical instrument 1 so that it can rotate around its longitudinal axis 15.

The previously described medical instrument 1, which is illustrated in FIGS. 1 and 2, is handled as follows:

In the starting position shown in FIGS. 1 and 2, in which the gripping members 4 and 5 of the handle 6 are essentially arranged horizontally and parallel to one another, as is shown in the rear view as seen in FIG. 2, the operator grips the medical instrument 1 in order, for instance, to grip a surgical needle and hold it by means of the tool 7. For this purpose the operator presses together the gripping members 4 and 5 of the handle 6, gripped with one hand, until the jaw members 7a and 7b are closed.

Because the gripping members 4 and 5 are pretensioned in the open position by the spring element 2, the operator must hold the gripping members 4 and 5 firmly grasped to keep from loosening the posture of the tool 7. With more extended tasks, the gripping members 4 and 5 of the handle 6 here exert a constant pressure point on the user's palm surfaces, which can result in fatigue and even the formation of blisters.

To alter the position of the gripping members 4 and 5 in his hand, without at the same time being obliged to release the grip on the handle 6, the operator can rotate the rotatable portion 14 of the gripping member 4, which portion is enclosed by the fingers of the hand that holds it, around the longitudinal axis 15 of the gripping member 4, as seen in FIGS. 3 and 4, so that the position of the gripping member 5 on the operator's palm surface is also modified and thus brings relief to the hand that does the holding.

In addition to relieving the hand, the fact that at least one portion 14 of the gripping member 4 can rotate allows the operator, at every hand position, always to adjust the gripping members 4 and 5 of the handle 6 with respect to one another in such a way as to ensure the best possible power transmission by the holding hand to the medical instrument 1.

The angle of rotation alpha of the rotatable gripping member 14 can be restricted by stops 19 to prevent excessive turning or slipping of the gripping member 4 in the holding hand.

A medical instrument 1 configured in this manner is distinguished in that the operator can modify the position of the gripping members 4 and 5 of the handle 6 to relieve the holding hand without releasing the gripping members 4 and 5.

1. A medical gripping and/or cutting instrument with a shaft having on its distal end a tool consisting of at least two jaw members and on its proximal end a handle consisting of at least two gripping members, and wherein at least one jaw member of the tool, for opening and closing, can be displaced with respect to the other jaw member by means of a rotatably configured gripping member of the handle, distinguished in that at least one portion of at least one gripping member of the handle can rotate around the longitudinal axis of this gripping members.

2. A medical gripping and/or cutting instrument according to claim 1, distinguished in that one gripping member of the handle is of bipartite construction, consisting of a rigid portion and a rotatable portion, in such a way that the rotatable portion can rotate at least partially around the rigid portion.

3. A medical gripping and/or cutting instrument according to claim 1, distinguished in that gripping indentations are configured on the rotatable portion of the gripping member.

4. A medical gripping and/or cutting instrument according to claim 1, distinguished in that the angle of rotation (alpha) of the rotatable portion can be restricted by stops.

5. A medical gripping and/or cutting instrument according to claim 1, distinguished in that the gripping members of the handle are pretensioned in the opened position by a spring element.
